(19) United States
(12) Patent Application Publication
(10) Pub. No.: US 2016/0076019 A1
(43) Pub. Date: Mar. 17, 2016

(54) SYSTEM AND METHOD FOR SONIC RADIATION FOR INFLUENCING CELLULAR STRUCTURES

(71) Applicant: STRATHSPEY CROWN HOLDINGS, LLC, Newport Beach, CA (US)

(72) Inventors: Robert Edward Grant, Laguna Beach, CA (US); David Haydn Mordaunt, Los Gatos, CA (US); Matthew T. Case, Laguna Hills, CA (US)

(21) Appl. No.: 14/488,101

(22) Filed: Sep. 16, 2014

(51) Int. Cl. C12N 13/00; A61N 7/00; C12Q 3/00; C12M 1/42; C12M 1/36; A61H 23/00; A61N 5/00

(52) U.S. Cl. CPC C12N 13/00; A61H 23/00; A61N 7/00; A61N 5/00; C12M 35/04; C12M 41/48; C12Q 3/00; A61H 2230/655

(57) ABSTRACT

A system and method for the present invention requires use of a generator, in combination with a radiation unit, to radiate acoustic waveform energy onto a target tissue (i.e. a cellular structure). During radiation of the target tissue in accordance with a predetermined titration-like protocol, the influence of the waveform energy on the cellular structure is periodically monitored. The protocol is stopped when the cellular structure has been transformed or morphed into a desired phenotype.

### FIELD OF THE INVENTION

The present invention pertains generally to systems and methods for causing a transformational or morphological change in a cellular structure using waveform energy radiation. More specifically, the present invention pertains to systems and methods which epigenetically influence cellular structures with waveform energy radiation, wherein the radiation frequency is equal to or near the natural frequency of the cellular structure being radiated. The present invention is particularly, but not exclusively useful for systems that use sonic waves to alter the resultant functionality of in vivo or in vitro target tissues.

### BACKGROUND OF THE INVENTION

It is well known that sonic waves can have many different effects on tissue; for many different reasons. Moreover, in some cases, the consequences of sonically radiating tissue may be permanent.

Apart from applications where sonic waves may be used to induce or suggest changes in a particular mood or state of mind, it is also recognized that sonic waves can also be employed to cause transformative or morphological changes in cellular structure. Not surprisingly, many of these changes may be very beneficial. Thus, within the medical community there is increasing interest insofar as the extent to which such changes may be employed to beneficially alter the functionality of a cellular structure.

With the above in mind, consider the specific case of sonic waves and the effects they can have on tissue cells (i.e. a cellular structure). From a physical perspective, a tissue cell can be likened to a mechanical system. From this perspective it is also to be appreciated that the pressure of a sound wave is the result of the fluctuation (i.e. vibrational) component of the wave in its transmission medium (e.g. air). The importance of these physical observations is that sound (acoustic) pressure acts to exert a force against tissue (mechanical system). And this will happen whenever a sonic wave is incident on the tissue. Thus, the tissue will be influenced by the externally applied forces that are associated with the sound wave. As implied above, this influence will manifest itself on the tissue.

As a mechanical system, each individual cellular structure (tissue cell) has a natural frequency at which it will oscillate if it is not subjected to a continuous or repeated external force (i.e. when it is not damped). Cellular structures, however, are naturally damped. Thus, in response to an externally applied impulse force, amplitudes of the cellular structure's damped vibrational response to this force will progressively diminish. This will be the case unless the cellular structure is somehow otherwise subsequently stimulated, such as when a sustained vibratory frequency is applied to the cellular structure. Most noticeably, there will be no such diminution when the vibratory frequency is at or near the natural frequency of the cellular structure. Instead, in this case, a resonance condition is established wherein the vibratory nature of the cellular structure's response is sustained, and possibly even amplified.

From a biological perspective, each cell type (e.g. a liver cell) will have observable characteristics which naturally result from the cell's environment. A set of these observable characteristics is generally referred to as a phenotype. Further, it is known that the set of characteristics for a defined phenotype of a cellular structure can be epigenetically influenced by externally applied forces. Moreover, this can happen regardless whether the cellular structure is influenced in vivo or in vitro.

In light of the above, it is an object of the present invention to provide a system and method for using a radiation of waveform energy to epigenetically influence tissue cells, to thereby alter the functionality of an in vivo, or an in vitro, target tissue. It is another object of the present invention to provide a system and method for using a radiation of waveform energy to influence a change in target tissue by tuning a sonic wave to a frequency that is at or near the natural frequency of the target tissue. Still another object of the present invention is to provide a system and method for radiating waveform energy, in accordance with a predetermined titration-like protocol, to epigenetically influence tissue cells for the transformation or morphology of the target tissue into a desired phenotype. Yet another object of the present invention is to provide a system and method for using a radiated waveform energy to epigenetically influence tissue cells which is easy to use and commercially cost effective.

### SUMMARY OF THE INVENTION

The present invention pertains generally to the transformational or morphological change of cellular tissue under the influence of waveform energy radiation. From an engineering perspective, it is well known that waveform energy radiation creates forces (i.e. exerts pressure) on an object when the radiation is incident on the object. Further, it is also well known that these external forces can cause changes to tissue structure. The present invention is based on this interactive phenomenon.

For purposes of the present invention, the target tissue of interest may be any in vivo or in vitro cellular structure of the human body. It may be an individual cell, or it may be a group of cells together within the intercellular tissue (matrix) that supports the cells. As envisioned for the present invention, target tissue may also be an identifiable structure inside a cell, such as a chromosome. In each case, it is important to appreciate that as a mechanical structure, the cellular structure of a target tissue will have a unique natural frequency.

An initial consideration for implementation of the present invention is the task of defining a desired phenotype for the outcome. For example, the objective of a protocol for the present invention may be the creation of a particular type of stem cell (e.g. liver cell) from an otherwise undefined or undifferentiated cell. In this case, the desired phenotype (outcome) will be defined to have the requisite characteristics of the particular type stem cell that is desired (e.g. liver cell). As another example, the objective of a protocol may be to terminate the viability of a cellular structure, such as by killing cancer cells. Other examples can be cited. In each instance, however, and regardless of the specific outcome that is desired, the present invention employs waveform energy radiation for the purpose of epigenetically influencing a target tissue for its transformation or morphological change into a structure that corresponds to the desired phenotype.

As envisioned for the present invention, the radiation to be employed for influencing target tissue may be of any waveform energy known in the art. It may be electromagnetic radiation in the spectrum between wavelengths of 10⁻²⁵ m to 10³ m. It may also be periodic mechanical vibrations. In this latter case, the radiation may be acoustic sound waves in the range between 20 Hz and 20 kHz, and may also include infrasound waves (<20 Hz) and ultrasound waves (>20 kHz). Further, the radiation may be either continuous or pulsed, and the tone of the radiation may be either pure (single frequency) or complex (multi-frequency).

Structurally, a system for using a radiation of waveform energy to influence cellular structures within a target tissue will include a combination of various components. These include: components for generating and directing the radiation onto the target tissue; components for monitoring the target tissue; and a computer for controlling the generator and the radiation unit in accordance with a predetermined protocol.

In detail, the generator is used for generating the particular waveform energy radiation that is necessary to influence the target tissue. For this purpose it is important that the radiation be characterized by operational parameters having respective values which are established relative to the natural frequency of the target tissue. At a minimum, these operational parameters will include a frequency f and a volume intensity level v for the radiation, as well as a time duration t_{d} during which the target tissue is to be radiated. A radiation unit, which is incorporated with the generator, may include optics that are used for directing the radiation electromagnetic radiation (e.g. lasers) onto the target tissue and the cellular structure. Specifically, all of this is done in accordance with a predetermined protocol that is designed to epigenetically influence the target tissue and the cellular structure that may be within the target tissue. In a preferred embodiment of the present invention the radiation unit will be positioned at a distance d from the target tissue. Typically, the distance d will be greater than 10 millimeters (d>10 mm).

As indicated above, control over the system during the conduct of a protocol is managed by a computer. To do this, a device is provided for monitoring a phenotypic response of the target tissue and the cellular structure during the protocol. As envisioned for the present invention, this monitoring function can be performed by an appropriate sensor, or by the periodic performance of a biopsy. In the event, management and control of the protocol by the computer is terminated when the phenotypic response corresponds with the desired phenotype.

A method in accordance with the present invention begins by identifying the target tissue to be influenced (including the cellular structure), and by defining a desired phenotype for the target tissue. A natural frequency for the phenotype can then be determined by reference to the literature. It is then necessary to establish values for the operational parameters (e.g. p, v and t_{d}) that will properly characterize the radiation that is to be used. In particular, it is desirable to establish operational values that are operationally relative to the natural frequency of the target tissue (cellular structure). In detail, with knowledge of this natural frequency, the radiation frequency f can be set to resonate, or partially resonate, with the cellular structure that is to be influenced during conduct of the protocol.

Operationally, once parameters have been established for the radiation, the radiation can be directed onto the target tissue in accordance with a predetermined protocol. As noted above, the purpose here is to epigenetically influence the target tissue and the cellular structure. During the protocol, the target tissue is then monitored in a titration-like process to detect a phenotypic response from the target tissue and the cellular structure. The protocol is terminated when the phenotypic response corresponds with the desired phenotype.

In an embodiment of the present invention the radiation can be pulsed. For this embodiment, each radiation pulse will have a predetermined time duration t_{d} within a predetermined time interval tᵢ. Specifically, tᵢ will extend between the successive beginnings of respective radiation pulses (i.e. tᵢ>t_{d}).

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of components for a system in accordance with the present invention;

FIG. 2 is a time line of radiation pulses in a representative pulse train of waveform energy radiation in accordance with the present invention;

FIG. 3 is an illustration of the sequential progression of epigenetic influence on two different cellular structures during the transformation of the respective cellular structure into a desired phenotype; and

FIG. 4 is a flow chart of the interactive tasks involved in the methodology of the present invention.

### DESCRIPTION OF THE PREFERRED EMBODIMENTS

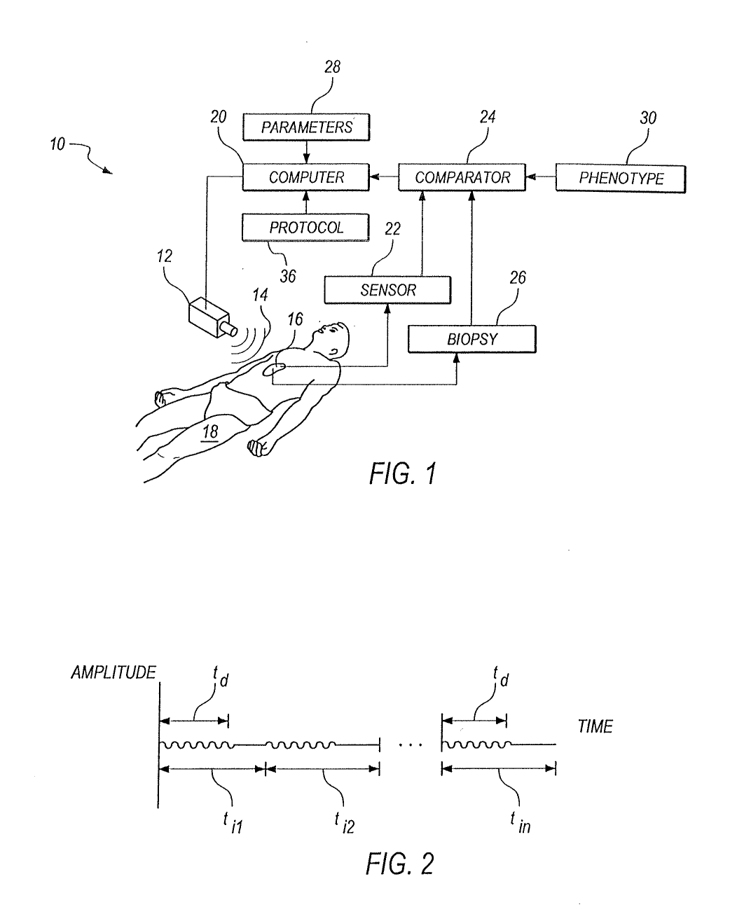

Referring initially to FIG. 1 a system in accordance with the present invention is shown and is generally designated 10. As shown, the system 10 is to be used for radiating waveform energy to epigenetically influence cellular structures within a target tissue. To do this, the system 10 includes a unit 12 for directing radiation 14 toward a target tissue 16 of a patient 18. In particular, it is envisioned that the unit 12 will be capable of generating a waveform energy radiation 14 that spans the electromagnetic spectrum of wavelengths between 10⁻²⁵ m and 10³ m. Further, it is envisioned that the radiation 14 may also include acoustic sound waves in the range between 20 Hz and 20 kHz, as well as infrasound waves (<20 Hz) and ultrasound waves (>20 kHz). In the case of sound waves, the energy waveform of radiation 14 may be either a pure frequency or a complex frequency and, in the case of electromagnetic waves, the radiation 14 may have either a single wavelength λ, or a combination of different wavelengths. Also, as envisioned for the present invention, the target tissue 16 may be either in vivo as shown in FIG. 1, or it may be in vitro.

Still referring to FIG. 1, it will be seen that the system 10 includes a computer 20 which is connected with the unit 12. Depending on the particular application for system 10, the computer 20 may perform various functions during a same protocol. For instance, in addition to providing operational details for the radiation 14, the computer 20 may also be used to operationally control movements of the unit 12.

As also shown in FIG. 1, the system 10 also includes a sensor 22 which is used to monitor the target tissue 16, and to transfer information pertaining to the target tissue 16 to a comparator 24. For this purpose, the sensor 22 can be of any type well known in the pertinent art that is capable of epigenetically monitoring a transformation or morphology of the target tissue 16. For example, sensor 22 may be employed to perform titration-like methodologies with processes such as bioelectrical impedance analysis and quantitative Polymerase Chain Reaction (PCR) techniques. The results of the monitoring performed by sensor 22 are then provided as input to the comparator 24. In the system 10, the comparator 24 is connected with the computer 20.

For an alternative to the use of a sensor 22 as disclosed above, it will be understood and appreciated by the skilled artisan that an epigenetic change (transformation/morphology) in the target tissue 16 can also be monitored by performing periodic biopsies 26 of the target tissue 16. Again, a titration methodology can be employed. In the event, the particular protocol which is used, its periodicity, and the extent to which the biopsy(ies) 26 is/are employed will be established on a case-by-case basis by the user of the system 10.

In addition to the hardware components for the system 10 mentioned above, various inputs for these components are required for an operation of the system 10. Importantly, the parameters 28 that are required for establishing the waveform energy of radiation 14 are a primary consideration. In particular the parameters 28 will necessarily include a selected frequency f for the vibration of the sound wave in the radiation 14. Also included will be the intensity level v for the max peak amplitudes of the sound wave, and a predetermined time duration t_{d} for the radiation 14. Depending on the particular application, the time duration t_{d} for the radiation 14 may be either continuous or pulsed.

Referring to FIG. 2, time considerations for the radiation 14 are shown. If the radiation 14 is to be pulsed during an operation of the system 10, each radiation pulse will continue for a predetermined time duration t_{d} within a predetermined time interval tᵢ. For a train of pulses (e.g. an n number of pulses as shown in FIG. 2), the predetermined time interval tᵢ for each individual pulse can be established to extend between the successive beginnings of respective radiation pulses in the train (i.e. tᵢ>t_{d}). Stated differently, each pulse will have a length tᵢ (tᵢ=time interval), during which the radiation 14 will be generated for the time duration t_{d}. On the other hand, for a continuous radiation 14, t_{d} will equal tᵢ (i.e. t_{d}=tᵢ and n=1).

Insofar as the frequency f of the radiation 14 is concerned, several considerations are possible. For one, as noted above, the frequency f may be pure or complex. For another, during a radiation 14, the predetermined frequency f may be alternated between a first frequency f₁ and a different second frequency f₂ (i.e. f₁≠f₂). Further, alternation of the frequencies may be set to occur at a predetermined repetition rate.

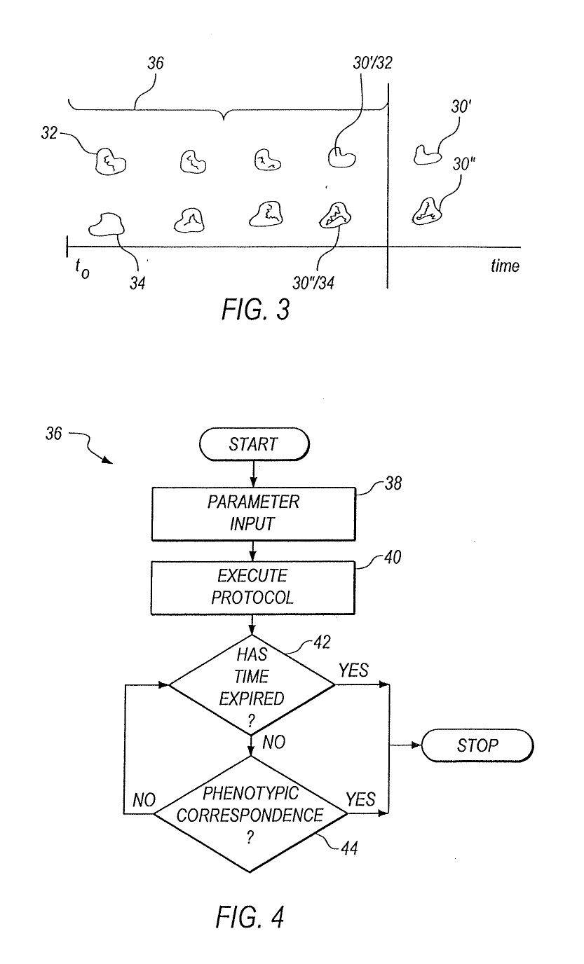

In an operation of the present invention, it is necessary for there to first be a determination and an identification of a desired phenotype 30. By definition, as used for the present invention, a phenotype 30 is set of observable characteristics of an individual resulting from its interaction with the environment. Here, reference to the word “individual” in the definition is taken to mean a cellular structure, a contiguous group of cellular structures, or a portion of a cellular structure, such as a chromosome. For the present invention, the cellular structure is alive and can be either in vivo or in vitro. With this in mind, consider the exemplary cellular structures 32 and 34 shown in FIG. 3.

For the examples presented here with reference to FIG. 3, consider the cellular structure 32 to be a cancer cell, and the cellular structure 34 to be an undifferentiated cell. The consequence on these respective cellular structures will then depend on the particulars of the protocol 36 that is employed for influencing a particular target tissue 16 with a particular radiation 14. Consider first, the transformation/morphology desired for an active cancer cell (cellular structure) 32. In this instance, the desired phenotype 30′ will be a cancer-free cellular structure. Importantly, once the desired phenotype 30′ has been identified, and defined, its definitional parameters 28 (including its natural frequency) must be input into the comparator 24. Depending on the characteristics of the desired phenotype 30′, operational parameters 28 for the radiation 14 (i.e. f, v, t_{d}, n and tᵢ) are established. Specifics of the particular protocol 36 that are required to influence cellular structure 32 into the desired phenotype 30′ are then followed and monitored.

In detail, during the conduct of a protocol 36, the sensor 22 (biopsy 26) is used to observe the cellular structure 32, and the comparator 24 is used to compare the cellular structure 32 with the desired phenotype 30′. Thus, the comparator 24 effectively monitors the transformation/morphology of the cellular structure 32 as it is being influenced by the radiation 14. When the comparator 24 determines a cellular structure 30′/32 has been created which corresponds with the desired phenotype 30′ (i.e. a cancer-free cell), the protocol 36 can be terminated.

For another example, consider the transformation/morphology of a cellular structure such as an undifferentiated cell 34. In this case, the desired phenotype 30″ may be selected from any of various particular type cells (e.g. a liver cell). As with the earlier example, definitional parameters 28 for a desired phenotype 30″ are input into the comparator 24. Also, the required parameters 28 for radiation 14 are established, and an appropriate protocol 36 is followed. As before, when the comparator 24 determines a cellular structure 30″/34 has been created which corresponds with the desired phenotype 30″ (i.e. a liver cell), the protocol 36 can be terminated.

For the conduct of a typical protocol 36, refer to FIG. 4. There it will be seen that block 38 requires parameter input for an operation of the system 10. Based on the above disclosure, it will be appreciated that this parameter input is really a two-step process. First, a desired phenotype (e.g. phenotype 30′ or 30″) needs to be identified and defined. Importantly, this includes selecting a natural frequency for the desired phenotype 30′ or 30″. Most often this can be accomplished by selecting a natural frequency from previously compiled empirical data. Second, the parameters 28 for operating the radiation unit 12 need to be established (i.e. f, v, t_{d}, n and tᵢ).

Once system 10 has been set for operation as described above, block 40 indicates that the protocol 36 can be performed. The actual conduct of the protocol 36, however, is very event-dependent and may vary considerably depending on the transformation/morphology desired for a particular target tissue 16. Moreover, due to the titration-like methodology that is envisioned by the present invention for a protocol 36, and the many variables that are involved, the actual conduct of a protocol 36 must necessarily be essentially under the purview of the user of the system 10. Accordingly, any time requirements for the protocol 36 that are to be maintained (see inquiry block 42), and a determination of phenotypic correspondence that is indicative of operational completion (see inquiry block 44), are effectively dependent on operational judgments of the user.

While the particular System and Method for Sonic Radiation for Influencing Cellular Structures as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for using a radiation of waveform energy to epigenetically influence cellular structures within a target tissue which comprises the steps of:
identifying the target tissue including the cellular structure;
determining a natural frequency for the target tissue;
defining a desired phenotype for the target tissue;
generating the radiation, wherein the radiation is characterized by operational parameters;
establishing values for each operational parameter of the radiation, wherein the values are established relative to the natural frequency of the target tissue;
directing the radiation onto the target tissue in accordance with a predetermined protocol to epigenetically influence the target tissue and the cellular structure;
monitoring a phenotypic response of the target tissue and the cellular structure; and
terminating the protocol when the phenotypic response corresponds with the desired phenotype.

2. A method as recited in claim 1 wherein operational values for the radiation are established to provide a predetermined fluence of the radiation on the target tissue.

3. A method as recited in claim 1 wherein the predetermined protocol comprises the steps of:
tuning the radiation to a selected frequency f,
adjusting the peak values of amplitudes of radiation to a selected intensity level v; and
providing a predetermined time duration t_{d} for activation of the generating step.

4. A method as recited in claim 3 wherein the radiation is continuous during the time duration t_{d}.

5. A method as recited in claim 3 wherein the tuning step is accomplished by alternating the predetermined frequency between a first frequency f₁ and a different second frequency f₂ (f₁≠f₂).

6. A method as recited in claim 5 wherein alternation of the frequencies occurs at a predetermined repetition rate.

7. A method as recited in claim 3 wherein the tuning step is accomplished by simultaneously tuning to a plurality of different frequencies.

8. A method as recited in claim 3 wherein the radiation is pulsed during the generating step, with each radiation pulse having radiation of the predetermined time duration t_{d} within a predetermined time interval tᵢ between the successive beginnings of respective radiation pulses (i.e. tᵢ>t_{d}).

9. A method as recited in claim 1 further comprising the steps of:
focusing the radiation onto the target tissue using a radiation unit; and
positioning the radiation unit at a distance d from the target tissue, wherein the distance d is greater than 10 millimeters (d>10 mm).

10. A method as recited in claim 1 wherein the waveform energy is selected from the group consisting of mechanical vibrations and electromagnetic radiation.

11. A method as recited in claim 1 wherein the target tissue is selected from the group consisting of in vivo tissue and in vitro tissue.

12. A method as recited in claim 1 wherein the monitoring step is accomplished in a titration-like process selected from the group consisting of bioelectrical impedance analysis and quantitative Polymerase Chain Reaction (PCR) techniques.

13. A method as recited in claim 1 wherein the radiation is sonic and is selected from the group consisting of a pure frequency and a complex frequency.

14. A method for epigenetically influencing a cellular structure in a target tissue which comprises the steps of:
defining a desired phenotype for a target tissue, wherein the desired phenotype includes the cellular structure and has a characteristic natural frequency;
radiating the target tissue with waveform energy having operational parameters established relative to the natural frequency of the target tissue, wherein the radiation of the waveform energy is accomplished in accordance with a predetermined protocol to epigenetically influence the target tissue and the cellular structure;
monitoring a phenotypic response of the target tissue and the cellular structure; and
terminating the protocol when the phenotypic response corresponds with the desired phenotype.

15. A method as recited in claim 14 wherein the predetermined protocol further comprises the steps of:
tuning the radiation to a selected frequency f;
adjusting the peak values of amplitudes of radiation to a selected intensity level v; and
providing a predetermined time duration t_{d} for activation of the generating step.

16. A method as recited in claim 14 wherein the waveform energy is sonic radiation, including infrasound and ultrasound wave and is selected from the group consisting of a pure frequency and a complex frequency.

17. A method as recited in claim 14 wherein the target tissue is selected from the group consisting of in vivo tissue and in vitro tissue, and wherein the monitoring step is accomplished in a titration-like process selected from the group consisting of bioelectrical impedance analysis and quantitative Polymerase Chain Reaction (PCR) techniques.

18. A system for using a radiation of waveform energy to epigenetically influence cellular structures within an in vivo target tissue, which comprises:
a generator for generating the radiation to influence the target tissue including the cellular structure, wherein the target tissue has a natural frequency, and wherein the radiation is characterized by operational parameters having respective values established relative to the natural frequency of the target tissue;
a radiation unit incorporated with the generator for directing the radiation onto the target tissue and the cellular structure in accordance with a predetermined protocol to epigenetically influence the target tissue and the cellular structure;
a device for monitoring a phenotypic response of the target tissue and the cellular structure; and
a computer for controlling the generator and the radiation unit in accordance with a predetermined protocol, and for terminating the protocol when the phenotypic response corresponds with a desired phenotype.

19. A system as recited in claim 18 wherein the waveform energy is sonic radiation, including infrasound and ultrasound wave and is selected from the group consisting of a pure frequency and a complex frequency.

20. A system as recited in claim 18 wherein the target tissue is selected from the group consisting of in vivo tissue and in vitro tissue, and wherein the monitoring step is accomplished in a titration-like process selected from the group consisting of bioelectrical impedance analysis and quantitative Polymerase Chain Reaction (PCR) techniques.
